(19) United States
(12) Patent Application Publication
(10) Pub. No.: US 2008/0146534 A1
(43) Pub. Date: Jun. 19, 2008

(54) PHARMACEUTICAL COMPOSITION FOR REDUCING THE RISKS ASSOCIATED WITH CARDIOVASCULAR AND CEREBROVASCULAR DISEASES

(76) Inventors: San-Laung Chow, San Jose, CA (US); David Wong, Milpitas, CA (US)

Correspondence Address: PATTERSON & SHERIDAN, L.L.P., 3040 POST OAK BOULEVARD, SUITE 1500, HOUSTON, TX, 77056, US

(21) Appl. No.: 11/956,488

(22) Filed: Dec. 14, 2007

Related U.S. Application Data
(60) Provisional application No. 60/870,082, filed on Dec. 14, 2006.

(51) Int. Cl. A61K 31/397; A61K 31/47; A61P 9/10

(52) U.S. Cl. 514/210.02; 514/312

(57) ABSTRACT

Methods and compositions enable treating atherosclerosis and atherosclerotic related diseases and/or lowering the risk of developing atherosclerosis related diseases. The composition includes an antilipemic agent combined with a vasodilator and/or an inhibitor of platelet aggregation for treating cardiovascular and cerebrovascular related diseases. In one embodiment, a pharmaceutical composition includes ezetimibe and cilostazol and is administered to treat patients suffering from cardiovascular and cerebrovascular related diseases and/or offer protection to those at risk to develop atherosclerosis and related diseases.

### CROSS-REFERENCE TO RELATED APPLICATIONS

This application claims benefit of U.S. provisional patent application Ser. No. 60/870,082, filed Dec. 14, 2006, which is herein incorporated by reference.

### BACKGROUND OF THE INVENTION

It is believed that high levels of cholesterol in the blood stream are prone to induce many diseases and ailments. In general, cholesterols can be made by the liver or absorbed from food consumption. There are many types of drugs to lower the levels of cholesterol in the blood stream.

Statin drugs reduce low density lipoprotein (LDL, bad) cholesterol levels and treat high cholesterol levels by interrupting the formation of cholesterol from the circulating blood. The statins are grouped together as antilipemic agents by the PDR (Physicians' Desk Reference). Lipemia is an excess of lipid (fat) in the blood, so an antilipemic agent acts to reduce lipids. Hyperlipidemia is a general term for elevated concentrations of any or all lipids in the plasma.

In general, statin drugs improve cholesterol levels primarily by inhibiting a liver enzyme called HMG CoA reductase, and statin drugs are also known as HMG-CoA reductase inhibitors, among the different types of antilipemic agents. Because of their effectiveness and they are generally well tolerated, they have become some of the most commonly prescribed drugs in the United States. Exemplary statins include atorvastatin, fluvastatin, lovastatin, pravastatin, rosuvastatin calcium, and simvastatin.

Fibrates represent another type of cholesterol-reducing drug. The fibrate medications lower the levels of fats (lipids) in the blood, including cholesterol and triglycerides, and are also effective in raising high density lipoprotein (HDL, good cholesterol) in the blood. Ezetimibe also lowers cholesterol by blocking the absorption or re-absorption of cholesterol from the intestine. Other types of cholesterol-reducing drugs to treat high cholesterol levels include bile acid sequestrants and nicotinic acid.

Atherosclerosis occurs when cholesterols, fatty substances, cellular waste products, calcium and other substances deposit in artery walls, resulting in hardening of the arteries and narrowing of their interior diameter. Cholesterol contributes to atherosclerosis, which can cause cardiovascular and cerebrovascular diseases. Coronary arteries supply blood and oxygen to the heart muscle such that atherosclerosis of the coronary arteries may lead to angina and raise the risk of heart attacks. Carotid and vertebral arteries supply blood and oxygen to the brain such that atherosclerosis of the carotid and vertebral arteries may increase the risk of strokes.

Lowering the cholesterol, especially low density lipoprotein (LDL, bad) cholesterol, helps to prevent or reverse atherosclerosis and may decrease the risk of heart attacks. However, almost one third of patients in primary and secondary prevention programs treated with one or more cholesterol-lowering agents fail to reach target LDL levels. There is also a residual cardiovascular morbidity observed in clinical trials in spite of cholesterol lowering treatments.

Therefore, a need exists for treatments that lower the risks for cardiovascular and cerebrovascular diseases and decrease the risks of morbidity.

### SUMMARY OF THE INVENTION

Embodiments of the invention provide a method and a composition for preventing and treating atherosclerosis and atherosclerotic related diseases and/or lowering the risk of developing cardiovascular and cerebrovascular related diseases. The composition includes an antilipemic agent combined with a vasodilator and/or an inhibitor of platelet aggregation for treating cardiovascular and cerebrovascular related diseases. A drug combination of one embodiment comprises an antilipemic agent in combination with a vasodilator for treating, preventing, and/or reducing the risk of developing atherosclerosis and atherosclerotic disease events. Cilostazol and ezetimibe respectively provide examples of the vasodilator and the antilipemic agent. For one embodiment, a pharmaceutical composition includes ezetimibe and cilostazol and is administered to treat patients suffering from cardiovascular and cerebrovascular related diseases and/or offer protection to those at risk to develop atherosclerosis and related diseases.

### DETAILED DESCRIPTION

Embodiments of the invention relate to use of an antilipemic agent together with a vasodilator to prevent and treat atherosclerosis and atherosclerotic related diseases and/or lower the risk of developing cardiovascular and cerebrovascular related diseases. A pharmaceutical composition is provided and includes the antilipemic agent for lowering the level of cholesterols and a vasodilator for improving the flow of blood and oxygen. The antilipemic agent is useful in modifying lipid levels in the blood stream and reducing the levels of low density lipoprotein cholesterols.

The antilipemic agent may include a cholesterol lowering agent, such as 3-hydroxy-3-methylglutaryl-coenzyme A (HMG-CoA) reductase inhibitors, statins, niacin, fibrates, ezetimibe, bile acid sequestrants, cholesterol ester transfer protein inhibitors, and combinations thereof. Dosages of the antilipemic agent vary depending on types of the antilipemic agent used. For example, ezetimibe can be used at a daily dose of about 10 mg/day with a cholesterol-lowering diet.

Statins may offer potential benefits for reducing the incidence of strokes and improving the prognosis of stroke. Examples of suitable statins include atorvastatin, fluvastatin, lovastatin, pravastatin, rosuvastatin calcium, and simvastatin. Bezafibrates, as pan-peroxisome proliferator activated receptor activators, offer beneficial pleiotropic effects related to glucose metabolism and insulin sensitivity. Ezetimibe represents an anti-hyperlipidemic medication which acts by decreasing cholesterol absorption in the intestine. Ezetimibe can be used as the antilipemic agent alone or together with statins (e.g. ezetimibe/simvastatin). Ezetimibe localizes at the brush border of the small intestine, where ezetimibe inhibits the absorption of cholesterol from the diet. Specifically, ezetimibe appears to bind to the Niemann-Pick C1-Like 1 (NPC1L1) protein on the gastrointestinal tract epithelium, a critical mediator of cholesterol absorption. In addition to this direct effect, decreased cholesterol absorption leads to an increase in LDL-cholesterol uptake into cells, thus decreasing levels in the blood plasma.

The vasodilator may include a compound that relaxes the smooth muscle in blood vessels, which causes the blood vessels to dilate. For example, cilostazol (6-[4-(1-cyclohexyl-1H-tetrazol-5-yl)butoxy]-3,4-dihydro-2(1H)-quinolinon) may provide the vasodilator and is a drug used for the treatment of intermittent claudication which involves leg pain after a long walk. Intermittent claudication refers to a condition caused by narrowing of the arteries that supply the legs with blood. Patients with intermittent claudication develop pain when they walk because not enough oxygen-containing blood reaches the active leg muscles. Cilostazol reduces the pain of intermittent claudication by dilating the arteries, thereby improving the flow of blood and oxygen to the legs. Cilostazol allows people with intermittent claudication to exercise longer before developing their characteristic leg pain and to walk longer before they must stop because of the pain. While the mechanism of action of cilostazol is not clear, cilostazol is a vasodilator and inhibitor of platelet aggregation as it is an inhibitor of phosphodiesterase III (PDE III). Cilostazol appears to inhibit PDE III at clinical doses, causes a modest increase in heart rate (about 7 beats/minute) and a small increase in ventricular premature beat (VPB) rates (from 1/hour to 4/hour). Cilostazol reversibly inhibits platelet aggregation induced by a variety of stimuli, including thrombin, adenosine diphosphate (ADP), collagen, arachidonic acid, epinephrine, and shear stress. Cilostazol can be used at doses of about 100 mg/day to about 200 mg/day. Other suitable examples of the vasodilator with PDE III activity include milrinone, vesnarinone, enoximone, pimobendan, and flosequinan.

A combinational therapy can be advantageous since the vasodilator helps to increase blood flow and/or inhibits platelet aggregation whereas the use of the antilipemic agent, such as statins, ezetimibe, fibrates, and/or niacin, regulates serum lipids. Different antilipemic agents help to regulate the levels of serum lipid and cholesterol by different mechanisms such that one or more can be selected on the basis of their safety and effectiveness to offer desirable benefits in patients when combined with the use of the vasodilator. Without being limited to any particular theory or mechanism of action, it is believed that dilation to increase arterial diameter and blood flow further inhibits deposition of cholesterol, already reduced in level due to the antilipemic agent, along the arterial walls. Further, blood vessel dilation helps open restrictions where cardiac events can originate and increase blood flow to, when in combination with reduced cholesterol levels due to the antilipemic agent, both facilitate removal of cholesterol from the arterial walls and inhibit deposition relative to slower flowing blood.

One embodiment of the invention provides the combination of ezetimibe and cilostazol in a pharmaceutical composition, such as a solid or liquid oral dosage form, to treat or lower the risk of developing atherosclerosis and atherosclerotic disease events. Applying both medications together in individual dosing achieves such desirable benefits. A pharmaceutical composition containing both drug entities enables treatment of patients suffering from cardiovascular and cerebrovascular related diseases, offers protection for those at risk to develop atherosclerosis and related diseases, and/or helps to reduce the side effects associated with each medication.

### EXAMPLES

### Example 1

Ezetimibe, cilostazol, microcrystalline cellulose, lactose and magnesium stearate are mixed and compressed directly into tablet.

### Example 2

Ezetimibe, cilostazol, microcrystalline cellulose, and lactose are granulated with a binder in a high-shear mixer. The granules are dried, milled, and blended with magnesium stearate to provide a final mixture. The final mixture is compressed into a tablet.

### Example 3

Ezetimibe, cilostazol, microcrystalline cellulose, lactose and a gelling water-soluble polymer (e.g., hydroxymethyl cellulose) are granulated in a high-shear mixer. The granules are dried, milled and blended with other lubricants (e.g., magnesium stearate) to provide a final mixture. The final mixture is compressed into a sustained release hydrogel tablet.

### Example 4

Ezetimibe, cilostazol, swelling polymer(s) and other ingredients are pelletized and spherized into beads. Then, the beads are encapsulated in capsules.

### Example 5

A core tablet of ezetimibe and cilostazol is made as described in Example 1 or Example 2 and is coated with a water-semi-permeable membrane to form a film-coated extended release dosage form.

### Example 6

Ezetimibe beads are blended with cilostazol beads and encapsulated into capsules. Their relative strengths are determined by a ratio of the amount of these two beads.

While the foregoing is directed to embodiments of the present invention, other and further embodiments of the invention may be devised without departing from the basic scope thereof, and the scope thereof is determined by the claims that follow.

What is claimed is:

1. A pharmaceutical composition, comprising:
an antilipemic agent for lowering cholesterol level of a patient; and
a vasodilator that is an inhibitor of phosphodiesterase III.

2. The pharmaceutical composition of claim 1, wherein the antilipemic agent comprises ezetimibe.

3. The pharmaceutical composition of claim 1, wherein the vasodilator comprises cilostazol.

4. The pharmaceutical composition of claim 1, wherein the vasodilator includes at least one of milrinone, vesnarinone, enoximone, pimobendan, and flosequinan.

5. The pharmaceutical composition of claim 1, wherein the antilipemic agent comprises a combination of cholesterol lowering drugs.

6. The pharmaceutical composition of claim 1, wherein the antilipemic agent comprises a statin.

7. The pharmaceutical composition of claim 1, wherein the antilipemic agent comprises niacin.

8. The pharmaceutical composition of claim 1, wherein the antilipemic agent comprises ezetimibe and a statin.

9. The pharmaceutical composition of claim 1, wherein the antilipemic agent includes at least one of a statin, niacin, a fibrate, ezetimibe, and a bile acid sequestrant.

10. A pharmaceutical composition, comprising:
a single dosage form having an antilipemic agent together with cilostazol.

11. The pharmaceutical composition of claim 10, wherein the antilipemic agent comprises ezetimibe.

12. The pharmaceutical composition of claim 10, wherein the single dosage form is a tablet.

13. The pharmaceutical composition of claim 10, wherein the single dosage form is a solid for oral delivery and the antilipemic agent comprises ezetimibe.

14. The pharmaceutical composition of claim 10, wherein the single dosage form is a capsule containing beads of the antilipemic agent and the cilostazol.

15. The pharmaceutical composition of claim 10, wherein the antilipemic agent comprises a combination of cholesterol lowering drugs.

16. The pharmaceutical composition of claim 10, wherein the antilipemic agent comprises a statin.

17. A method of treating atherosclerosis, comprising:
administering an antilipemic agent to a patient such that the antilipemic agent is effective for lowering cholesterol level of the patient; and
administering a vasodilator to the patient such that the vasodilator is effective at inhibiting phosphodiesterase III in the patient.

18. The method of claim 17, wherein the antilipemic agent comprises ezetimibe.

19. The method of claim 17, wherein the vasodilator comprises cilostazol.

20. The method of claim 17, wherein the vasodilator comprises cilostazol and the antilipemic agent comprises ezetimibe.
